(19) United States
(12) Patent Application Publication
(10) Pub. No.: US 2023/0326127 A1
(43) Pub. Date: Oct. 12, 2023

(54) MEDICAL IMAGE PROCESSING METHODS AND SYSTEMS FOR ANALYSIS OF CORONARY ARTERY STENOSES

(71) Applicants: Singapore Health Services Pte Ltd, Singapore (SG); Agency For Science, Technology And Research, Singapore (SG)

(72) Inventors: Liang ZHONG, Singapore (SG); Weimin HUANG, Singapore (SG); Junmei ZHANG, Singapore (SG); Gaurav CHANDOLA, Singapore (SG); Soo Teik LIM, Singapore (SG); Ru San TAN, Singapore (SG); Swee Yaw TAN, Singapore (SG)

(73) Assignees: Singapore Health Services Pte Ltd, Singapore (SG); Agency For Science, Technology And Research, Singapore (SG)

(21) Appl. No.: 18/023,108

(22) PCT Filed: Aug. 26, 2021
(86) PCT No.: PCT/SG2021/050510

§ 371 Date: Feb. 24, 2023

(30) Foreign Application Priority Data
Aug. 26, 2020 (SG) 10202008205U

(51) Int. Cl. G06T 7/10; G06T 7/62; G06T 7/64; G06T 7/66; G06V 10/46; G06T 17/00; G06T 7/00

(52) U.S. Cl. CPC G06T 17/00; G06T 7/0012; G06T 7/10; G06T 7/62; G06T 7/64; G06T 7/66; G06V 10/46; G06T 2207/10081; G06T 2207/30101

(57) ABSTRACT

Methods and systems for simulating a stent in a coronary artery lumen structure are disclosed. A method of simulating a stent in a coronary artery lumen structure, the method comprises: reconstructing a three-dimensional coronary artery tree from segmented coronary lumen contours; replacing part of the three-dimensional artery tree with a candidate stent structure; and simulating pressure distribution through the coronary artery tree to determine a non-invasive fractional flow reserve through the candidate stent structure.

### CROSS REFERENCE

The present application is a national phase entry under 35 U.S.C. § 371 of International Application No. PCT/SG2021/050510, filed Aug. 26, 2021, published in English, which claims the benefit of the filing date of Singapore Patent Application No. 10202008205U, filed Aug. 26, 2020, the disclosures of which are incorporated herein by reference.

### TECHNICAL FIELD

The present disclosure relates to medical image processing methods for analysis of coronary artery stenoses, and in particular to simulation of stents.

### BACKGROUND

Coronary artery disease develops when atherosclerotic plaques build up inside the coronary artery, leading to stenosis in lumen, which reduces blood supply to the myocardium. Revascularization of coronary stenosis, including percutaneous coronary intervention (PCI), may become necessary when the stenosis results in ischemia.

Although the usage of invasive fractional flow reserve (FFR) measurement is recommended, its usage is low due to a variety of factors, including cost, time and the potential adverse effects associated with the invasive FFR measurement. Its application is also challenging for bifurcation and tandem lesions. We have adopted a system to assess non-invasive FFR, named as FFR_{B}, by combining both computed tomography coronary angiography (CTCA) and reduced-order computational fluid dynamics (CFD) techniques. Based on the platform, we propose to model virtual stenting for assessing the functional status of the coronary lesions post-PCI so that the operator can review and compare different implantation strategies and their impact on coronary physiology post-PCI. This will enhance appropriate patient selection for invasive treatment strategy, and individualized stent selection and deployment planning, potentially transforming clinical decision-making and treatment planning in coronary artery disease.

FFR is the gold standard to select the patients who are likely to benefit from the revascularization and assess the functional improvement post-revascularization. However, FFR is measured during invasive coronary angiography (ICA) at hyperaemic state induced by the administration of adenosine, adenosine 5′-triphosphate or papaverine. To measure FFR, a pressure-sensor-tipped wire is inserted into the target diseased coronary artery via the catheter. The pressures distal and proximal to the stenosis are recorded and their ratio used to quantify FFR. For coronary artery disease patients with lesions FFR >0.8, medical therapy is recommended. Coronary revascularization is advocated for those lesions with FFR ≤0.8. Post-revascularization FFR >0.8 signifies procedural success. Although FFR-guided coronary revascularization has been shown to enhance clinical outcomes, FFR measurements are not widely used in clinical practice [5] due to the high medical cost, additional procedure time and potential complications involved in the invasive procedure. A non-invasive methodology is needed to discriminate the ischemic lesion and assess the outcome of revascularization.

In terms of revascularization, PCI has proven to be effective for restoring the blood flow to heart for coronary artery disease patients. The global coronary stents market at 5.2 billion US dollar in 2017 is projected to increase to 8.4 billion US dollars by 2025. Decisions for implantation location, stents selection (e.g., size/type/number) and stent implantation strategies remain complex and challenging, especially when treating bifurcation and tandem lesions, which are prone to in-stent restenosis (ISR), stent thrombosis and the associated adverse clinical events. Although use of FFR measurement is recommended during PCI for treating bifurcation lesions, it is challenging when re-crossing the pressure wire through the struts of the first implanted stent in main vessel. For tandem lesions, it is challenging to assess the severity of individual stenoses with FFR measurements, due to physiological interplay. Therefore, a tool to model the functional status of the coronary lesions after PCI with different implantation strategies is needed to aid PCI treatment planning.

CTCA is a valuable non-invasive test to assess coronary artery disease based on high-resolution anatomical depiction of the lesions. It has high sensitivity and low specificity, however, for discriminating ischemic coronary lesions. Combining CTCA and CFD modelling of intracoronary hemodynamics has made non-invasive assessment of the functional significance of coronary lesions feasible. An example of a non-invasive method FFR_{B} using reduced order CFD modelling and novel outlet boundary conditions is set out in the following publication: Zhang JM, Zhong L, Luo T, Lomarda AM, Huo Y, Yap J, Lim ST, Tan RS, Wong ASL, Tan JWC, Yeo KK, Fam JM, Keng FYJ, Wan M, Su B, Zhao X, Allen JC, Kassab GS, Chua TSJ, Tan SY. Simplified models of non-invasive fractional flow reserve based on CT images. PLOS ONE 2016;11(5):e0153070 (DOI:10.1371/journal.pone.0153070).

### SUMMARY

According to a first aspect of the present disclosure, a method of simulating a stent in a coronary artery lumen structure is provided. The method comprises: reconstructing a three-dimensional coronary artery tree from segmented coronary lumen contours; replacing part of the three-dimensional artery tree with a candidate stent structure; and simulating pressure distribution through the coronary artery tree to determine a non-invasive fractional flow reserve through the candidate stent structure.

In an embodiment, the method further comprises identifying a candidate location for the candidate stent structure.

In an embodiment, identifying the candidate location for the candidate stent structure comprises: determining a mean lumen area as a function of straightened length of a vessel; identifying a proximal point of a lesion and a distal point of the lesion from the mean lumen area as a function of straightened length of the vessel; and determining the candidate stent location from the proximal point of the lesion and the distal point of the lesion.

In an embodiment, identifying the proximal point of the lesion and the distal point of the lesion from the mean lumen area as a function of straightened length of the vessel comprises determining a curvature of the mean lumen area as a function of straightened length of the vessel and identifying the proximal point of the lesion and the distal point of the lesion as maxima in the absolute values of the curvature.

In an embodiment, identifying the proximal point of the lesion and the distal point of the lesion from the mean lumen area as a function of straightened length of the vessel comprises determining a change of slope of the mean lumen area as a function of straightened length of the vessel and identifying the proximal point of the lesion and the distal point of the lesion as maxima of the change of slope.

In an embodiment, identifying the proximal point of the lesion and the distal point of the lesion from the mean lumen area as a function of straightened length of the vessel comprises identifying a location of a minimum in the mean lumen area and identifying the proximal point of the lesion and the distal point of the lesion on respective sides of the minimum in mean lumen area.

In an embodiment, the method further comprises determining a diameter for the candidate stent structure from the mean lumen diameter at the proximal point of the lesion and the distal point of the lesion.

In an embodiment, the method further comprises determining a length for the candidate stent structure from the location from the proximal point of the lesion and location of the distal point of the lesion.

In an embodiment, the method further comprises adding an extension allowance to the length for the candidate stent structure.

In an embodiment, the method further comprises segmenting a coronary artery lumen structure from a set of computed tomography coronary angiography images to obtain the segmented coronary lumen contours.

In an embodiment segmenting the coronary artery lumen structure from a set of computed tomography coronary angiography images to obtain the segmented coronary lumen contours comprises: designating points at aortic sinus as starting points of coronary artery trees; determining vessel centerlines for arteries of the artery trees; using the centerlines to create a stretched multiplanar reformatted volume for segments of the artery trees; extracting longitudinal cross sections from the stretched multiplanar reformatted volume;

detecting lumen borders in the extracted longitudinal cross sections; and detecting lumen border contours in slices of the multiplanar reformatted volume using the detected lumen borders.

In an embodiment, the cross sections are extracted from the stretched multiplanar reformatted volume at 45 degree intervals.

In an embodiment, the vessel centerlines are determined using a Hessian filter.

According to a second aspect of the present disclosure, a computer readable carrier medium carrying processor executable instructions which when executed on a processor cause the processor to carry out a method as set out above.

According to a third aspect of the present disclosure, a medical image processing system for simulating a stent in a coronary artery lumen structure is provided. The medical image processing system comprises: a processor and a data storage device storing computer program instructions operable to cause the processor to: reconstruct a three-dimensional coronary artery tree from segmented coronary lumen contours; replace part of the three-dimensional artery tree with a candidate stent structure; and simulate pressure distribution through the coronary artery tree to determine a non-invasive fractional flow reserve through the candidate stent structure.

In an embodiment, the data storage device further stores computer program instructions operable to cause the processor to: identify a candidate location for the candidate stent structure.

In an embodiment, the data storage device further stores computer program instructions operable to cause the processor to identify the candidate location for the candidate stent structure by: determining a mean lumen area as a function of straightened length of a vessel; identifying a proximal point of a lesion and a distal point of the lesion from the mean lumen area as a function of straightened length of the vessel; and determining the candidate stent location from the proximal point of the lesion and the distal point of the lesion.

In an embodiment, identifying the proximal point of the lesion and the distal point of the lesion from the mean lumen area as a function of straightened length of the vessel comprises determining a curvature of the mean lumen area as a function of straightened length of the vessel and identifying the proximal point of the lesion and the distal point of the lesion as maxima in the absolute values of the curvature.

In an embodiment, identifying the proximal point of the lesion and the distal point of the lesion from the mean lumen area as a function of straightened length of the vessel comprises determining a change of slope of the mean lumen area as a function of straightened length of the vessel and identifying the proximal point of the lesion and the distal point of the lesion as maxima of the change of slope.

In an embodiment, identifying the proximal point of the lesion and the distal point of the lesion from the mean lumen area as a function of straightened length of the vessel comprises identifying a location of a minimum in the mean lumen area and identifying the proximal point of the lesion and the distal point of the lesion on respective sides of the minimum in mean lumen area.

In an embodiment, the data storage device further stores computer program instructions operable to: determine a diameter for the candidate stent structure from the mean lumen diameter at the proximal point of the lesion and the distal point of the lesion.

In an embodiment, the data storage device further stores computer program instructions operable to: determine a length for the candidate stent structure from the location from the proximal point of the lesion and location of the distal point of the lesion.

In an embodiment, the data storage device further stores computer program instructions operable to: add an extension allowance to the length for the candidate stent structure.

In an embodiment, the data storage device further stores computer program instructions operable to: segment a coronary artery lumen structure from a set of computed tomography coronary angiography images to obtain the segmented coronary lumen contours.

In an embodiment, the data storage device further stores computer program instructions operable to: segment a coronary artery lumen structure from a set of computed tomography coronary angiography images to obtain the segmented coronary lumen contours by: designating points at aortic sinus as starting points of coronary artery trees;
determining vessel centerlines for arteries of the artery trees; using the centerlines to create a stretched multiplanar reformatted volume for segments of the artery trees; extracting longitudinal cross sections from the stretched multiplanar reformatted volume;
detecting lumen borders in the extracted longitudinal cross sections; and detecting lumen border contours in slices of the multiplanar reformatted volume using the detected lumen borders.

In an embodiment, the cross sections are extracted from the stretched multiplanar reformatted volume at 45 degree intervals.

In an embodiment, the vessel centerlines are determined using a Hessian filter.

### BRIEF DESCRIPTION OF THE DRAWINGS

In the following, embodiments of the present invention will be described as nonlimiting examples with reference to the accompanying drawings in which:

FIG. 1 is a flowchart showing a method of simulating stenting in a patient according to an embodiment of the present invention;

FIG. 2 is block diagram showing a medical image processing system according to an embodiment of the present invention;

FIG. 3 is a flowchart showing a medical image processing method of simulating stenting according to an embodiment of the present invention;

FIG. 4A to FIG. 4D show a set of images illustrating respectively: a set of computed tomography coronary angiography (CTCA) images 410 of a patient; a multi-planar reformatted image derived from the CTCA images; lumen contour delineation in both longitudinal and transverse views; and a 3D reconstructed coronary artery model for the patient;

FIG. 5A to FIG. 5E illustrate an example of selecting stent parameters in a method according to an embodiment of the present invention.

FIG. 6A to FIG. 6D show a methodology to simulate deployment of a stent in an embodiment of the present invention;

FIG. 7A to FIG. 7D show a comparison of invasive FFR and non-invasive FFR_{B} for functional assessment of a stenosis;

FIG. 8 shows example of using methods according to embodiments of the present invention in a comparison of 3 different stenting strategies to treat a patient with tandem lesions in the left anterior descending artery;

FIG. 9A shows the correlation between FFR and FFR_{B} before stenting and FIG. 9B shows the corresponding Bland-Altman plot; and

FIG. 10A shows the correlation between FFR and FFR_{B} after stenting and FIG. 10B shows the corresponding Bland-Altman plot.

### DETAILED DESCRIPTION

The current disclosure relates to systems and methods to quantify the coronary artery stenosis, model the percutaneous coronary intervention (PCI) procedure and assess the hemodynamics before and after virtual stenting. The whole procedure includes coronary artery segmentation, centerline extraction, centerline tracking, cross sectional artery image generation, artery lumen segmentation, stenosis detection/quantification, shape-restoration to mimic the scenario with implanted stents (size, length and number), deployment, non-invasive fractional flow reserve (FFR_{B}) assessment before and after virtual stenting.

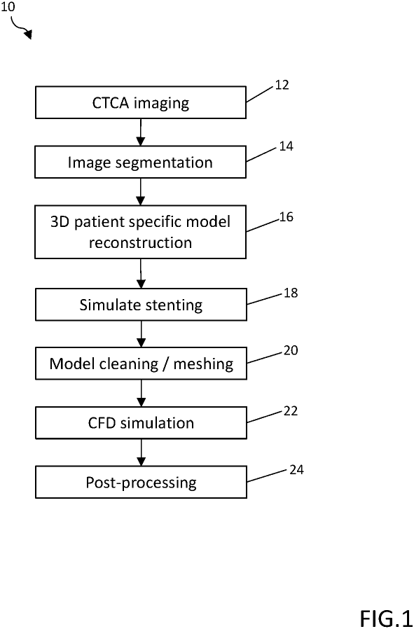

FIG. 1 is a flowchart showing a method of simulating stenting in a patient according to an embodiment of the present invention. The method 10 shown in FIG. 1 may be used to simulate the outcome of a stenting procedure on a patient.

In a CTCA imaging step 12, computed tomography coronary angiography image acquisition is carried out. The imaging is carried out according to the Society of Cardiovascular Computed Tomography guidelines. The CTCA imaging may be performed on contemporary multi-slice computed tomography scanners yielding high image spatial resolution such as the following: Toshiba Aquilion One 320 slice, Canon Aquilion ONE / Genesis 640, Siemens Somatom Force Dual Source and the Philips Brilliance iCT and others. Heart rate moderating beta-blockers or other drugs including ivabradine may be administrated to patients with resting heart rate >65 beats/minute, and sublingual glyceryl trinitrate may be administered prior to each scan. Prospective ECG-triggered scanning mode may be used for CTCA scans. All CTCA images may be saved in DICOM format.

In an image segmentation step 14, CTCA image processing is performed to segment the coronary artery lumen structure. The contour detection in the transverse slice is guided by the intersection points of each transverse slice with the longitudinal contours. The delineation of the lumen contours is supported in both the transversal and longitudinal planes. The segmented lumen contours are saved for the processing in the next step.

In a 3D patient specific model reconstruction step 16, all segmented coronary lumen contours are merged together to generate the patient-specific 3D reconstructed coronary artery model.

In a simulate stenting step 18, part of the 3D model of the patient’s coronary artery tree is replaced by a simulated stent. In some embodiments, a stenotic region of the coronary artery tree is detected and this stenotic region is replaced by the simulated stent. In other embodiments the stenotic region is manually selected by a user for replacement by the simulated stent. More than one stenotic region may be replaced by a stimulated stent.

Following the simulate stenting step 18, a model cleaning/meshing step 20 is carried out. In the model cleaning/meshing step 20, the 3D model is cleaned to have the inlet and outlet boundaries perpendicular to the respective vessel centerlines. Next, the computational domain of the cleaned-up model is discretized into tetrahedral, prisms or hexahedral shaped elements with boundary inflation layers.

Then, computational fluid dynamics (CFD) simulation 22 is carried out. The continuity and momentum-conservation (also known as the Navier-Stokes equations) equations for blood flow in the coronary arteries are solved for the computational domain using finite volume method, subject to patient-specific boundary conditions. Boundary conditions refer to the physiological conditions existing at the boundaries of the model being simulated. In our method, the inlet condition is set according to the mean brachial blood pressure of the patient. Outlet boundary conditions are specified by user defined functions which model the coronary microvasculature (parameterized using patient specific information). A no-slip boundary condition is set at the wall.

Following CFD simulation 22, a post processing step is carried out. In the post processing step 24, the pressure distribution on the coronary artery tree is used to calculate the non-invasive FFR_{B} values.

The method 10 shown in FIG. 1 may be used to aid in patient selection for stenting. The method may be carried out without the simulate stenting step 18 and following FFR_{B} calculation, a threshold of FFR_{B} ≤0.80 in any segment is used to discriminate ischemic condition and hence coronary revascularization can be considered. A FFR_{B} >0.80 is considered ischemia free and medical therapy is prescribed for these patients. These thresholds are based on extant medical knowledge in FFR applications, but these recommendations may be adjusted as future clinical experience accrues. For patients for which coronary revascularization is under consideration, the method 10 including the simulate stenting step 18 can be used to estimate the outcome of virtual stenting procedure. To compute the outcome of virtual stenting procedure, the 3D reconstructed coronary artery model is modified to incorporate a stent or stents at the location of the culprit lesion.

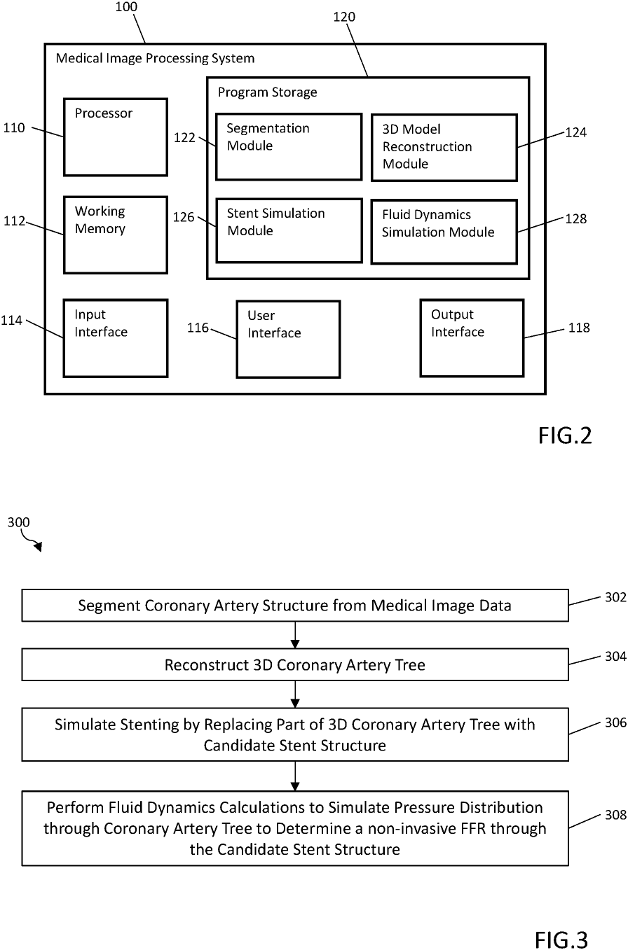

FIG. 2 shows a medical image processing system according to an embodiment of the present invention. The medical image processing system 100 is a computer system with memory that stores computer program modules which implement medical image processing methods according to embodiments of the present invention.

The medical image processing system 100 comprises a processor 110, a working memory 112, an input interface 114, a user interface 116, an output interface 118, program storage 120 and data storage 140. The processor 110 may be implemented as one or more central processing unit (CPU) chips. The program storage 120 is a non-volatile storage device such as a hard disk drive which stores computer program modules. The computer program modules are loaded into the working memory 112 for execution by the processor 110. The input interface 114 is an interface which allows data, such as patient computed tomography coronary angiography (CTCA) image data to be received by the medical image processing system 100. The input interface 114 may be a wireless network interface such as a Wi-Fi or Bluetooth interface, alternatively it may be a wired interface. The user interface 116 allows a user of the medical image processing system 100 to input selections and commands and may be implemented as a graphical user interface. The output interface 118 outputs data and may be implemented as a display or a data interface.

The program storage 120 stores a segmentation module 122, a 3D model reconstruction module 124, a stent simulation module 126, and a fluid dynamics simulation module 128. The computer program modules cause the processor 110 to execute various medical image processing which is described in more detail below. The program storage 120 may be referred to in some contexts as computer readable storage media and/or non-transitory computer readable media. As depicted in FIG. 2, the computer program modules are distinct modules which perform respective functions implemented by the medical image processing system 100. It will be appreciated that the boundaries between these modules are exemplary only, and that alternative embodiments may merge modules or impose an alternative decomposition of functionality of modules. For example, the modules discussed herein may be decomposed into sub-modules to be executed as multiple computer processes, and, optionally, on multiple computers. Moreover, alternative embodiments may combine multiple instances of a particular module or sub-module. It will also be appreciated that, while a software implementation of the computer program modules is described herein, these may alternatively be implemented as one or more hardware modules (such as field-programmable gate array(s) or application-specific integrated circuit(s)) comprising circuitry which implements equivalent functionality to that implemented in software.

FIG. 3 is a flowchart showing a medical image processing method of simulating a stent according to an embodiment of the present invention. The method 300 shown in FIG. 3 is carried out by the medical image processing system 100 shown in FIG. 2.

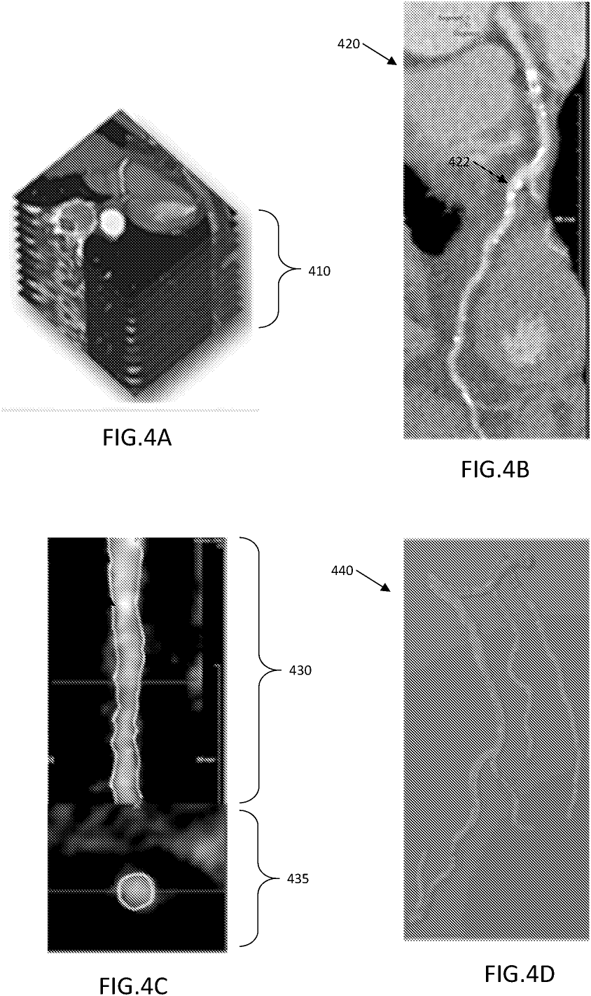

The method 300 is carried out on medical image data such as computed tomography coronary angiography (CTCA) image data of a patient. FIG. 4A shows a set of CTCA images 410 of a patient. As shown in FIG. 4A, the CTCA images 410 show a set of slices through the heart and surrounding region of the patient.

In step 302, the segmentation module 122 is executed by the processor 110 of the medical image processing system 100 to segment the coronary artery structure of the patient from the medical image data of the patient.

FIG. 4B shows a multi-planar reformatted image 420 derived from the CTCA images 410 shown in FIG. 4A. The multi-planar reformatted image 420 shows the coronary artery 422 of the patient.

FIG. 4C shows lumen contour delineation in both longitudinal and transverse views. The longitudinal view 432 shows the segmentation of the lumen 432 along its length and the transverse view 435 shows the segmentation of the lumen 435 in cross section.

CTCA image processing is performed to segment the coronary artery lumen structure. Points at the aortic coronary sinuses may be used to designate the starting points of the left and right coronary artery trees. Next, vessel centerline may be obtained using Hessian filter and vessel detection, or artificial intelligence (Al) methods, or in combination. Based on the centerline, a stretched multi-planar-reformatted (MPR) volume may be created for each segment of interest. Subsequently, 4 longitudinal cross sections, at 45° interval, may be extracted from the multiplanar reformatted MPR volume. Then, lumen borders in these 4 longitudinal images may be detected by a model-guided minimum cost approach (MCA). Similarly, lumen border contours may be detected in the transverse slice of the multiplanar reformatted MPR volume using MCA with a circular lumen model or other lumen models. Alternatively, as well as in combination, Al approaches can be adopted to generate the contours as well.

As described above, Al may be used as alternative to Hessian filter and vessel detection, and Al can be used as alternative to or in combination with MCA approach for contour delineation. Al methods for centerline extraction may include convolutional neural network as described in: Wolterink JM, van Hamersvelt RW, Viergever MA, Leiner T, Išgum I. Coronary artery centerline extraction in cardiac CT angiography using a CNN-based orientation classifier. Med Image Analysis 2019;51:46-60 and Ziqing Wan, Weimin Huang, Su Huang, Zhongkang Lu, Liang Zhong, Zhiping Lin. Coronary Artery Extraction from CT Coronary Angiography with Augmentation on Partially Labelled Data. Annu Int Conf IEEE Eng Med Biol Soc 2021.

Al methods used for coronary artery lumen segmentation may include deep learning framework using 3D U-Net: Weimin Huang, Lu Huang, Zhiping Lin, Su Huang, Yanling Chi, Jiayin Zhou, Junmei Zhang, Ru-San Tan, Liang Zhong. Coronary Artery Segmentation by Deep Learning Neural Networks on Computed Tomographic Coronary Angiographic Images. Annu Int Conf IEEE Eng Med Biol Soc 2018: 608-611, deep learning encoder-decoder such as Residual U-Net: Cheng Zhu, Xiaoyan Wang, et al. Cascaded residual U-net for fully automatic segmentation of 3D carotid artery in high-resolution multi-contrast MR images, Phys Med Biol 2021;66(4):045003, or other methods and/or combinations.

Returning now to FIG. 3, in step 304, the 3D model reconstruction module 124 is executed by the processor 110 of the medical image processing system 100 to reconstruct a 3D model of the coronary artery tree of the patient. All segmented coronary lumen contours are merged together to generate the patient-specific 3D reconstructed coronary artery model. We first construct a surface mesh model for each coronary artery and the branches, which is represented in the physical space, where the origin of the coordinates is defined by the computer tomographic scanning. The mesh is mapped back to the image space, where the origin of the coordinates is defined at (0,0,0) or the first voxel of the image volume. Any voxelization method can be used then to create the image voxels inside the mesh. The images containing each individual coronary artery and branches down to 1 mm (this is the current limit of resolution of the branches, but the limit of resolution may improve with imaging technology advancement) are added to form a 3D image containing all the coronary arteries. With this image, meshing operation such as marching cube may be used to create a new mesh that forms the surface mesh containing all coronaries. The process simplifies the mesh creation on the mesh level for a whole coronary tree, which requires handling the intersection between different meshes.

FIG. 4D shows a 3D reconstructed coronary artery model. As shown in FIG. 4D the 3D reconstructed coronary artery model 440 models the three-dimensional shape of the patient’s coronary artery tree.

In step 306, the stent simulation module 124 is executed by the processor 110 of the medical image processing system 100 to simulate stenting by replacing one or more parts of the three-dimensional coronary artery tree with a candidate stent structure. The critical parameters for deploying stent include the starting and ending points of the stent or stents, and each corresponding diameter and length.

In some embodiments step 306 comprises determining locations and dimensions of a candidate stent structure and then simulating stenting by replacing part or parts of the three-dimensional coronary artery tree with a candidate stent structure or multiple candidate stent structure.

In other embodiments, step 306 comprises receiving a user input indicating one or more candidate stent structures and locations for the candidate stent structures and then simulating stenting by replacing part or parts of the three-dimensional coronary artery tree with a candidate stent structure or multiple candidate stent structure.

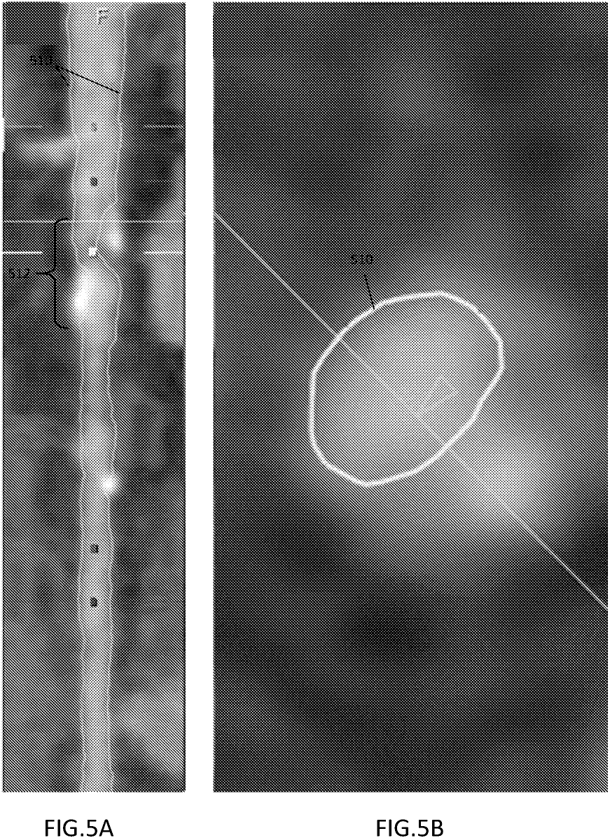

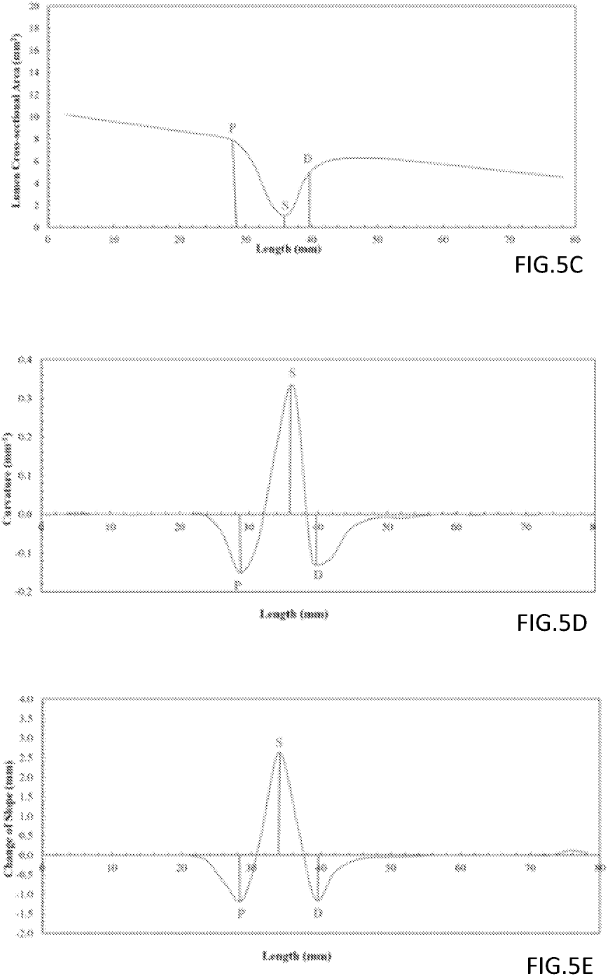

FIG. 5A to FIG. 5E illustrate an example of selecting stent parameters in a method according to an embodiment of the present invention.

FIG. 5A is a longitudinal view of coronary lumen contours. As can be seen in FIG. 5A the lumen contours 510 include a stenotic region 512 where the artery narrows.

FIG. 5B is a transverse view of coronary lumen contours. As shown in FIG. 5B, the lumen contours show the cross section of the artery at a point along its length. From this, the cross-sectional area of the lumen can be determined along the length of the lumen.

FIG. 5C is a graph of coronary lumen area along the stenosed coronary artery shown in FIG. 5A. The graph is plotted against the straightened length of the artery from proximal (left) to distal (right), which is derived from 3D curved multiplanar reformatting of the coronary artery along an automatic centerline. The curve is smoothed using a median or other low-pass filter to minimize the effect of noise. As shown in FIG. 5C, the coronary stenosis is maximal at a point “S” which corresponds to the minimum lumen area. A proximal point “P” corresponds to the proximal end of the stenosis and a distal point “D” corresponds to the distal end of the stenosis. The points “P” and “D” may be identified from maximal absolute values in either the curvature or the change in slope at either side of point “S”.

FIG. 5D is a graph showing the curvature of the graph shown in FIG. 5C. The curvature is defined as the reciprocal of the radius of an osculating circle and is computed using the independent coordinates method for planar curves as described in: Lewiner T, Gomes J, Lopes H, Craizer M. Curvature and torsion estimators based on parametric curve fitting. Computers and Graphics 2005;29:641-55.

As shown in FIG. 5D, the points “P” and “D” may be identified as the points to the left and right of point “S” with maximal absolute values of the curvature.

FIG. 5E is a graph showing the change in slope of the graph shown in FIG. 5C. The change of slope which is calculated as the difference of slopes for two adjacent points along the curve.

As shown in FIG. 5E, the points “P” and “D” may be identified as the points to the left and right of point “S” with maximal absolute values of the change in slope.

Using either the maximal absolute value of the curvature or the maximal absolute value of the change of slope, the two points “P” and “D” can be automatically determined separately by starting from “S” and then proceeding to the left and to the right, respectively. The distance from points “P” to “D” represents the minimum length of stenotic segment that needs to be replaced by a virtual stent. A user defined extension allowance (e.g. 2 to 3 mm) may be added to both ends of the “P” and “D” to ensure adequate landing zones and will allow some degree of freedom for commercial stent length selection within a range.

As can be seen from FIGS. 5C, 5D and 5E, the straightened lengths correspond to points “P” and “D” are 28.5 mm and 39.2 mm respectively. Considering the landing zone, the virtual stents will cover the region from straightened length at 27.6 mm to 42.6 mm, which corresponds to mean diameter (cross-sectional area) of 3.2 mm (8.04 mm²) and 2.8 mm (6.16 mm²) at the proximal and distal ends respectively.

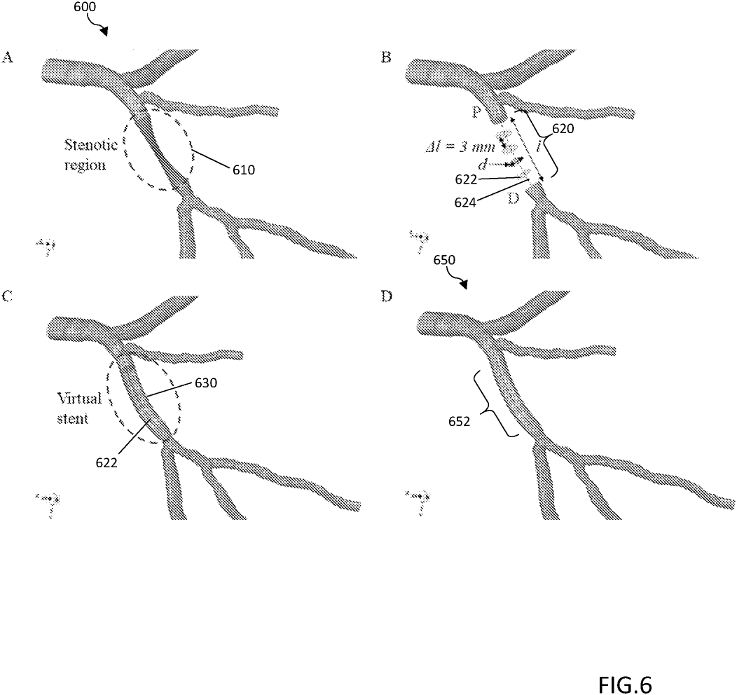

In the 3D model, we can search the planes corresponding to the area of 8.04 mm2 and 6.16 mm2 respectively to decide the starting and ending planes of the stent segment (this is shown in FIG. 6A). In addition, the distance between the proximal and distal ends is 15 mm and the average lumen diameter between these two points is 3.0 mm.

Current generation of commercial stents have nominal diameters and recommended balloon expansion limits due to their open-cell design. The diameter of the commercial stent chosen should have a range that spans the mean diameters at proximal and distal locations. Many coronary stents have open-cell design that allows for expansion beyond the nominal diameters. Depending on the stent type, the recommended maximum stent expansion limit may exceed 50% of nominal stent diameter.

The methodology of simulating deployment of a stent will now be described with reference to FIG. 6A to FIG. 6D.

FIG. 6A shows a patient specific 3D coronary artery model with a stenotic region. As shown in FIG. 6A, the coronary artery model 600 has a stenotic region 610 vessel with lesion along the left anterior descending artery.

FIG. 6B shows the generation of a simulated or virtual stent to replace the stenotic region. As shown in FIG. 6B, the proximal location P and the distal location D of the stenotic region are identified. These points may be identified according to the process described above with reference to FIG. 5A to FIG. 5E. For example, the cross-sectional area is first checked along the centerline of the vessel, to identify the planes “P” and “D”, which have cross-sectional areas close to those of “P” and “D” identified in FIG. 5C.The section 620 between the proximal location P and distal D location is removed from the model. A series of circular surfaces 622 are generated along and perpendicular to the original lumen centerline 624. These circular surfaces 622 have a distance of 3 mm between each other and diameter of 3 mm (the averaged mean diameters at proximal and distal locations).

FIG. 6C shows the simulated or virtual stent in the 3D coronary artery model. As shown in FIG. 6C, the circular surfaces 622 are interpolated with B-spline interpolation to form a virtual stent segment 630.

FIG. 6D shows a new model which incorporates the virtual stent segment. As shown in FIG. 6D the new model 650 has a section 652 which is formed from the virtual or simulated stent described above. The remainder of the new model 650 corresponds to the patient coronary artery model 600 shown in FIG. 6A.

Returning now to FIG. 3, in step 308, the fluid dynamics simulation module 128 is executed by the processor 110 of the medical image processing system 100 to simulate blood flow through the coronary artery tree including the virtual stent segment and determine a non-invasive FFR_{B}.

During the fluid dynamics simulation, the continuity and momentum-conservation (also known as the Navier-Stokes equations) equations for blood flow in the coronary arteries are solved for the computational domain using finite volume method, subject to patient-specific boundary conditions. Boundary conditions refer to the physiological conditions existing at the boundaries of the model being simulated. In our method, the inlet condition is set according to the mean brachial blood pressure of the patient. Outlet boundary conditions are specified by user defined functions which model the coronary microvasculature (parameterized using patient specific information). A no-slip boundary condition is set at the wall.

Following the same technique, additional virtual stents with different corresponding stent diameters and lengths can be “deployed” at multiple locations in the 3D coronary artery model. The operator can then interactively review the anatomical reconstruction of employing various stenting strategies that can differ in terms of stent number, location, length and diameter. Once the model with deployed stent is ready, non-invasive FFR_{B} after virtual stenting can be calculated. With this, the operator can review the physiological effects of the planned revascularization strategy.

As described above, the proximal and distal points, stent length, and stent diameter at each coronary lesion may be determined during the execution of the method. User definable allowances proximal and distal to the proximal and distal ends of the virtual stent (e.g. 2 to 3 mm, in order to secure adequate landing zones for the stent), respectively, as well as user definable allowance for stent diameter expansion (to simulate stent inflation beyond nominal diameter in clinical practice) can be manually input by user to generate one or more user virtual stents. CFD modelling can be performed to generate pressure and FFR_{B} maps for one or more virtual stent/s per coronary lesion.

In an embodiment of the proposed clinical application of the method, the user can select commercial stents that best fit the one or more virtual stents with favourable coronary hemodynamic properties. Commercial stents that approximate and are shorter and narrower than the selected virtual stent/s taking into account user defined virtual stent length and diameter allowances will be identified. The length and diameter of each selected commercial stent can then be fed into the model to simulate commercial stent implant, and CFD modelling and FFR_{B} map generated automatically. The placement of the stent can be automatically performed by centring the centre of the stent with the centre of the lesion segment. The user has option to make manual adjustment of virtual stent placement, with CFD modelling and FFR_{B} map generated automatically. Available as well as user selected commercial stent lengths, nominal diameters and diameter expansion limits with corresponding technical parameters of balloon expansion can be uploaded and updated into a library maintained by the proposed service and/or at user site.

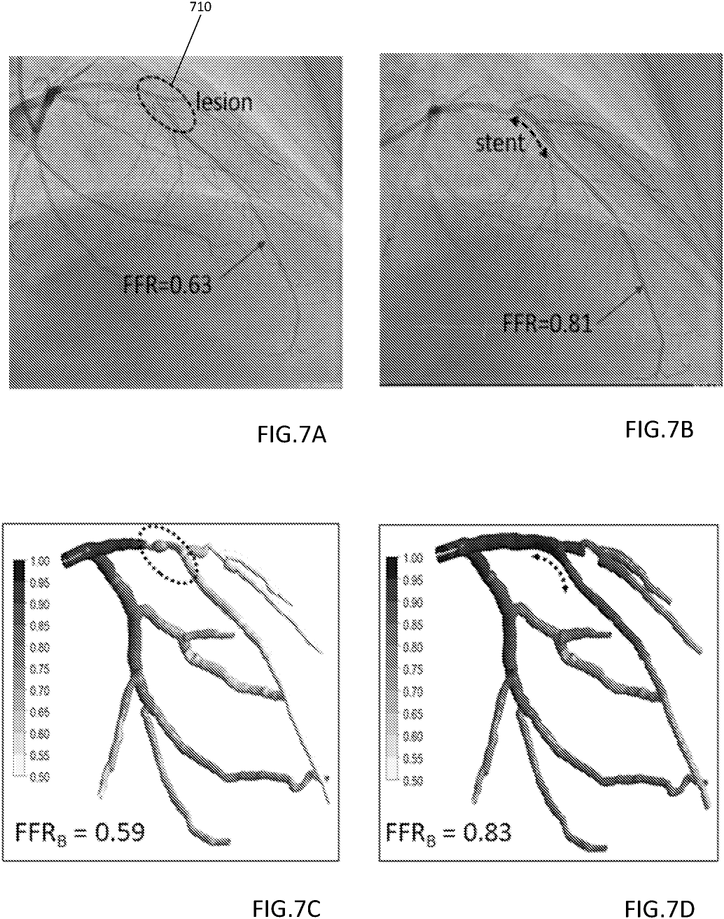

FIG. 7A to FIG. 7D show a comparison of invasive FFR and non-invasive FFR_{B} for functional assessment of a stenosis. This example demonstrates the application of FFR_{B} for pre-percutaneous coronary intervention (PCI) coronary artery disease diagnosis and post-PCI outcome prediction. As shown in FIG. 7A, the patient has a stenosis 710 in the left anterior descending coronary artery. The invasive FFR was measured as 0.63.

FIG. 7B shows the left anterior descending coronary artery with a stent on the vessel. Following the stenting procedure the measured invasive FFR value is 0.81.

FIG. 7C shows a modelled coronary artery tree for the patient prior to the stenting procedure. A functional significant stenosis is diagnosed at the left anterior descending coronary artery with FFR_{B} 0.59 (<0.80), a value that is close to the measured invasive FFR of 0.63.

FIG. 7D shows the predicted outcome of the stenting procedure using a method according to an embodiment of the present invention. The computed FFR_{B} post PCI is 0.83, which is close to the measured invasive FFR value of 0.81.

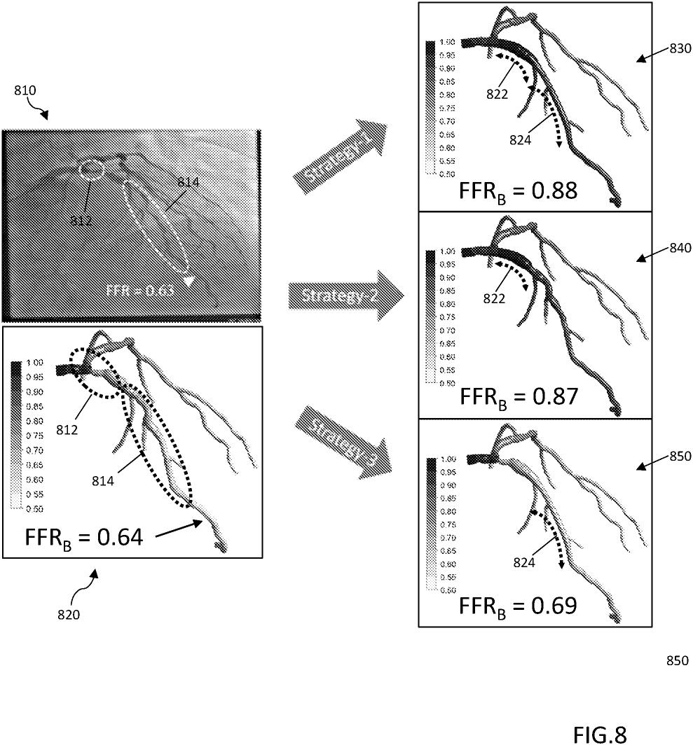

An example to demonstrate the application an embodiment of the present invention for comparing different stenting strategies for tandem lesions along the left anterior descending artery is shown in FIG. 8.

As shown in FIG. 8, an image 810 shows a first lesion 812 and a second lesion 814 along the left anterior descending artery. The invasive measured FFR is 0.63.

A three dimensional artery tree model 820 is generated for the patient. The model includes the first lesion 812 and the second lesion 814. From this model, the non-invasive FFR_{B} is calculated as 0.64.

Using the methods described above, three different stenting strategies are simulated. A first stenting strategy 830 involves applying a first stent 822 in the region of the first lesion 812 and a second stent 824 in the region of the second lesion 814. As shown in FIG. 8, the predicted non-invasive FFR_{B} is 0.88 for the first stenting strategy 830. A second stenting strategy 840 involves applying the first stent 822 in the region of the first lesion 812 and not applying a stent in the region of the second lesion 814. As shown in FIG. 8, the predicted non-invasive FFR_{B} is 0.87 for the second stenting strategy 840. A third stenting strategy 850 involves applying the second stent 824 in the region of the second lesion 814 and not applying a stent in the region of the first lesion 812. As shown in FIG. 8, the predicted non-invasive FFR_{B} is 0.69 for the second stenting strategy 850.

Based on the simulation of stenting strategies described above with reference to FIG. 8, a clinician may select the second strategy since it results in a significant improvement in the predicted non-invasive FFR_{B}, while adding an additional stent in the region of the second lesion 814 only provides a minimal improvement.

To validate the methodology, a proof-of-concept study was carried out. 18 patients with suspected or known coronary artery disease were recruited and underwent CTCA followed by invasive coronary angiogram with FFR measurements and subsequently PCI to treat hemodynamically significant lesions. FFR was measured both before and after stenting for a total of 21 vessels (22 lesions).

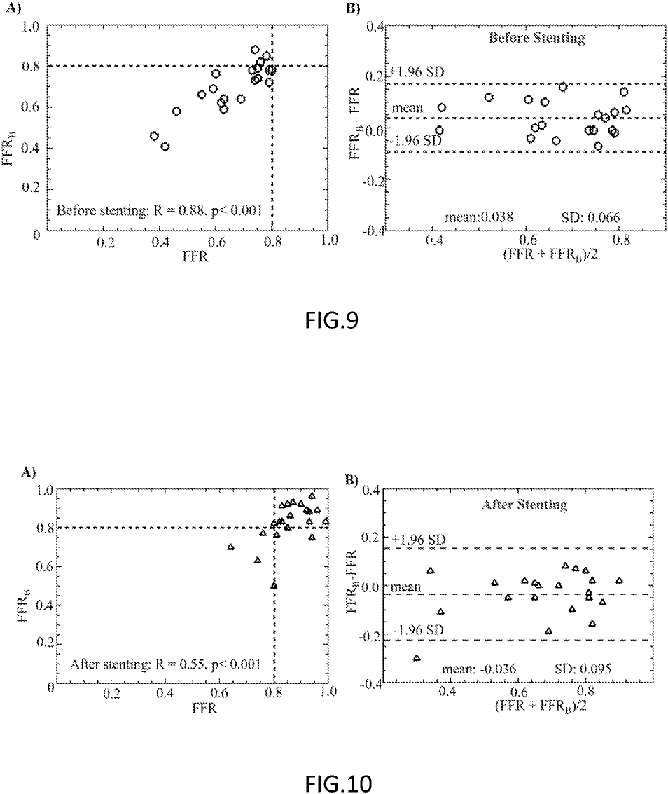

FIG. 9A shows the correlation between FFR and FFR_{B} before stenting and FIG. 9B shows the corresponding Bland-Altman plot.

FIG. 10A shows the correlation between FFR and FFR_{B} after stenting and FIG. 10B shows the corresponding Bland-Altman plot.

The calculated FFR_{B} showed an excellent correlation (R=0.88, p<0.001) with FFR before stenting and a fair correlation (R=0.55, p< 0.001) after stenting (FIG. 9A and FIG. 10A). The mean difference between FFR_{B} and FFR was 0.038 (95% limit of agreement: -0.09 to 0.17) before stenting (FIG. 9B) and -0.036 (95% limit of agreement: -0.22 to 0.15) after stenting (FIG. 10B). Diagnostic accuracy, sensitivity, specificity, positive predictive value (PPV) and negative predictive value (NPV) for FFR_{B} to compute residual ischemia post stenting, defined as a post stenting FFR_{B} ≤ 0.80, were 81%, 80%, 81%, 57%, and 93%, respectively.

Overall, FFR_{B} was a promising index to diagnose the hemodynamic significance of coronary stenosis, as well as compute the hemodynamic outcomes of the stenting procedure.

Coronary artery disease causes myocardial ischemia and contributes to 13% of deaths globally (projected to increase to 15% by 2030, World Health Organization 2011). PCI involving the implantation of intracoronary stents is an effective revascularization therapy to reduce ischemia in coronary artery disease. Decisions for stent selection (e.g., size /type/number), implantation location and strategies are challenging, especially when treating complex bifurcation and tandem lesions, which are prone to in-stent restenosis, late thrombosis and the associated adverse clinical events. Therefore, a tool to predict the functional status of the coronary lesions before and after PCI treatment will great help in management decision and procedure planning.

1. A method of simulating a stent in a coronary artery lumen structure, the method comprising:
reconstructing a three-dimensional coronary artery tree from segmented coronary lumen contours;
replacing part of the three-dimensional coronary artery tree with a candidate stent structure; and
simulating pressure distribution through the three-dimensional coronary artery tree to determine a non-invasive fractional flow reserve through the candidate stent structure.

2. A method according to claim 1, further comprising identifying a candidate location for the candidate stent structure.

3. A method according to claim 2, wherein identifying the candidate location for the candidate stent structure comprises:
determining a mean lumen area as a function of straightened length of a vessel;
identifying a proximal point of a lesion and a distal point of the lesion from the mean lumen area as a function of straightened length of the vessel; and
determining the candidate stent location from the proximal point of the lesion and the distal point of the lesion.

4. A method according to claim 3, wherein identifying the proximal point of the lesion and the distal point of the lesion from the mean lumen area as a function of straightened length of the vessel comprises determining a curvature of the mean lumen area as a function of straightened length of the vessel and identifying the proximal point of the lesion and the distal point of the lesion as maxima in absolute values of the curvature.

5. A method according to claim 3, wherein identifying the proximal point of the lesion and the distal point of the lesion from the mean lumen area as a function of straightened length of the vessel comprises determining a change of slope of the mean lumen area as a function of straightened length of the vessel and identifying the proximal point of the lesion and the distal point of the lesion as maxima of the change of slope.

6. A method according to claim 3, wherein identifying the proximal point of the lesion and the distal point of the lesion from the mean lumen area as a function of straightened length of the vessel comprises identifying a location of a minimum in the mean lumen area and identifying the proximal point of the lesion and the distal point of the lesion on respective sides of the minimum in mean lumen area.

7. A method according to claim 3, further comprising determining a diameter for the candidate stent structure from a mean lumen diameter at the proximal point of the lesion and the distal point of the lesion.

8. A method according to claim 3, further comprising determining a length for the candidate stent structure from a location of the proximal point of the lesion and a location of the distal point of the lesion.

9. (canceled)

10. A method according to claim 1, further comprising segmenting a coronary artery lumen structure from a set of computed tomography coronary angiography images to obtain the segmented coronary lumen contours.

11. A method according to claim 10, wherein segmenting the coronary artery lumen structure from a set of computed tomography coronary angiography images to obtain the segmented coronary lumen contours comprises:
designating points at aortic sinus as starting points of coronary artery trees;
determining vessel centerlines for arteries of the coronary artery trees;
using the vessel centerlines to create a stretched multiplanar reformatted volume for segments of the coronary artery trees;
extracting longitudinal cross sections from the stretched multiplanar reformatted volume;
detecting lumen borders in the extracted longitudinal cross sections; and
detecting lumen border contours in slices of the multiplanar reformatted volume using the detected lumen borders.

12. (canceled)

13. (canceled)

14. A computer readable carrier medium carrying processor executable instructions which when executed on a processor cause the processor to carry out a method according to claim 1.

15. A medical image processing system for simulating a stent in a coronary artery lumen structure, the medical image processing system comprising: a processor and a data storage device storing computer program instructions operable to cause the processor to:
reconstruct a three-dimensional coronary artery tree from segmented coronary lumen contours;
replace part of the three-dimensional coronary artery tree with a candidate stent structure; and
simulate pressure distribution through the three-dimensional coronary artery tree to determine a non-invasive fractional flow reserve through the candidate stent structure.

16. A medical image processing system according to claim 15, wherein the data storage device further stores computer program instructions operable to cause the processor to: identify a candidate location for the candidate stent structure.

17. A medical image processing system according to claim 16, wherein the data storage device further stores computer program instructions operable to cause the processor to identify the candidate location for the candidate stent structure by:
determining a mean lumen area as a function of straightened length of a vessel;
identifying a proximal point of a lesion and a distal point of the lesion from the mean lumen area as a function of straightened length of the vessel; and
determining the candidate stent location from the proximal point of the lesion and the distal point of the lesion.

18. A medical image processing system according to claim 17, wherein identifying the proximal point of the lesion and the distal point of the lesion from the mean lumen area as a function of straightened length of the vessel comprises determining a curvature of the mean lumen area as a function of straightened length of the vessel and identifying the proximal point of the lesion and the distal point of the lesion as maxima in absolute values of the curvature.

19. A medical image processing system according to claim 17, wherein identifying the proximal point of the lesion and the distal point of the lesion from the mean lumen area as a function of straightened length of the vessel comprises determining a change of slope of the mean lumen area as a function of straightened length of the vessel and identifying the proximal point of the lesion and the distal point of the lesion as maxima of the change of slope.

20. A medical image processing system according to claim 17, wherein identifying the proximal point of the lesion and the distal point of the lesion from the mean lumen area as a function of straightened length of the vessel comprises identifying a location of a minimum in the mean lumen area and identifying the proximal point of the lesion and the distal point of the lesion on respective sides of the minimum in mean lumen area.

21. A medical image processing system according to claim 17, wherein the data storage device further stores computer program instructions operable to: determine a diameter for the candidate stent structure from a mean lumen diameter at the proximal point of the lesion and the distal point of the lesion.

22. A medical image processing system according to claim 17, wherein the data storage device further stores computer program instructions operable to: determine a length for the candidate stent structure from a location of the proximal point of the lesion and a location of the distal point of the lesion.

23. (canceled)

24. A medical image processing system according to claim 15, wherein the data storage device further stores computer program instructions operable to: segment a coronary artery lumen structure from a set of computed tomography coronary angiography images to obtain the segmented coronary lumen contours.

25. (canceled)

26. (canceled)

27. (canceled)
